(19) United States
(12) Patent Application Publication
(10) Pub. No.: US 2015/0134059 A1
(43) Pub. Date: May 14, 2015

(54) DEFORMABLE ACCOMMODATIVE INTRAOCULAR LENS

(71) Applicant: COSTIN E. CURATU, CROWLEY, TX (US)

(72) Inventor: COSTIN E. CURATU, CROWLEY, TX (US)

(73) Assignee: Novartis AG, Basel (CH)

(21) Appl. No.: 14/079,754

(22) Filed: Nov. 14, 2013

(51) Int. Cl. A61F 2/16

(52) U.S. Cl. CPC A61F 2/1635
USPC 623/6.32; 623/6.37

(57) ABSTRACT

A deformable accommodating intraocular lens (IOL) is disclosed, where the IOL comprises a diffractive kinoform-like grating pattern on one or both of the anterior or posterior surfaces of the deformable IOL. The capsular bag of the eye exerts a distorting force on the IOL, changing its power and allowing for accommodation. The focal length variation obtained by the change in curvature of the refractive surface of the IOL is enhanced by the kinoform-like diffractive grating pattern in combination with the traditional refractive surface. The focal length variation obtained by stretching and shrinking of the diffractive pattern adds considerable power variation independent of the refractive index of the material used to make the IOL.

This application claims the priority of U.S. Provisional Patent Application No. 61/738,628 filed on Dec. 18, 2012.

### BACKGROUND

A human eye can be injured or diseased resulting in degeneration of or injury to the lens. The eye contains a capsular bag, which surrounds the natural lens. The capsular bag is transparent and holds and imparts shape to the lens. The eye's natural lens can adjust its focal length through a process known as accommodation, which is initiated by the ciliary body of the eye and effected by a series of zonular fibers. The zonular fibers are located in a relatively thick band around the lens and impart a force from the ciliary body to the capsular bag that can distort the lens and change its power.

Intraocular lenses (IOLs) are artificial lenses implanted in patients' eyes often in the capsular bag either to replace a patient's lens or, in the case of a phakic IOL, to complement the patient's lens. For example, the IOL may be implanted in place of the patient's lens during cataract surgery. Alternatively, a phakic IOL may be implanted in a patient's eye to augment the optical power of the patient's own lens. Single focal length IOLs have a single focal length or single power; thus, single focal length IOLs cannot accommodate, resulting in objects at a certain point from the eye being in focus, while objects nearer or further away being out of focus.

An improvement over the single focal length IOL is the accommodating IOL, which is made from a deformable material that can be compressed or distorted to adjust the power of the IOL over a certain range using the eye's natural zonular fibers and the ciliary body like a natural lens. Despite the advantages of accommodating IOLs, however, deformable accommodative lenses have relied mainly on change in the curvature of the lens in order to obtain power variation. This power variation is limited by the amount of curvature deformation and the refractive index of the IOL; however, the deformable materials used to make IOLs typically have a low index of refraction and thus power variation is limited. For example, FIG. 1 depicts a cross-sectional view of a conventional accommodative IOL 10. Note that when IOL 10 is compressed 11, the point of focus changes from 12 to 14, for a Δ_{f} 15.

Accordingly, what is needed is an accommodative lens that can achieve a greater power range than IOLs currently known in the art.

### BRIEF SUMMARY OF THE INVENTION

The method and system provide a deformable intraocular lens configured to deform in response to an ocular force to provide accommodation. The intraocular lens includes an anterior surface, a posterior surface disposed opposite the anterior surface, and a kinoform-like diffractive grating pattern disposed on at least one of the anterior surface or the posterior surface. The kinoform-like diffractive grating pattern varies under the ocular force. Enhanced power variation of the intraocular lens may thus be achieved.

### BRIEF DESCRIPTION OF THE DRAWINGS

FIG. 1 depicts a cross-sectional view of a conventional accommodative IOL.

FIG. 2 depicts an exemplary embodiment of an accommodative IOL according to the present invention.

FIGS. 3A through 3I depict cross-sectional views of nine different exemplary configurations for an accommodative IOL of the present invention.

FIGS. 4A through 4F depict cross-sectional views of six different exemplary configurations for an accommodative IOL of the present invention.

FIGS. 5A through 5D depict plan views of four of the exemplary configurations for selected accommodative IOLs from FIG. 2.

FIGS. 6A through 6F depict six exemplary diffraction patterns for use in the accommodative IOLs according to the present invention.

FIG. 7 is a flow chart depicting an exemplary embodiment of a method for using accommodative IOLs.

### DETAILED DESCRIPTION OF THE PREFERRED EMBODIMENTS

The exemplary embodiments relate to accommodative IOLs. The following description is presented to enable one of ordinary skill in the art to make and use the invention and is provided in the context of a patent application and its requirements. Various modifications to the exemplary embodiments and the generic principles and features described herein will be readily apparent. The exemplary embodiments are mainly described in terms of particular methods and systems provided in particular implementations. However, the methods and systems will operate effectively in other implementations. Phrases such as “exemplary embodiment”, “one embodiment” and “another embodiment” may refer to the same or different embodiments as well as to multiple embodiments. The embodiments will be described with respect to systems and/or devices having certain components. However, the systems and/or devices may include more or less components than those shown, and variations in the arrangement and type of the components may be made without departing from the scope of the invention. The exemplary embodiments will also be described in the context of particular methods having certain steps. However, the method and system operate effectively for other methods having different and/or additional steps and steps in different orders that are not inconsistent with the exemplary embodiments. Thus, the present invention is not intended to be limited to the embodiments shown, but is to be accorded the widest scope consistent with the principles and features described herein.

A number of embodiments of deformable accommodating IOLs are disclosed, where the IOLs comprise a diffractive kinoform-like grating pattern on one or both of the anterior or posterior surfaces of the IOL. The capsular bag of the eye exerts a distorting force on the IOL, changing its power and allowing for accommodation. The focal length variation obtained by the change in curvature of the refractive surface of the IOL is enhanced by the variation in the kinoform-like diffractive grating pattern as it is stretched or contracted in response to accommodation of the deformable IOL. The focal length variation obtained by stretching and shrinking of the diffractive pattern adds considerable power variation independent of the refractive index of the material used to make the IOL. The patterned IOLs of the present invention may have a meniscus configuration, a plano-convex configuration, a bi-convex configuration or any other configuration not inconsistent with the present invention.

An IOL including the kinoform-like diffractive grating of the invention may be thinner than IOLs without the kinoform-like diffractive grating due to the enhanced variation in power imparted by the stretching or shrinking of the kinoform-like diffractive grating. In addition, the diffractive power and power variation imparted by the kinoform-like diffractive grating is independent of the refractive index of the material from which the IOL is fashioned. A lens of high compressibility with a low refractive index may thus be used. The stretching and contraction or shrinking of the kinoform-like diffractive grating may be used to compensate for aberrations caused by deformation of the refractive surface during accommodation. Further, the kinoform-like diffractive grating may reduce longitudinal chromic aberrations of the eye.

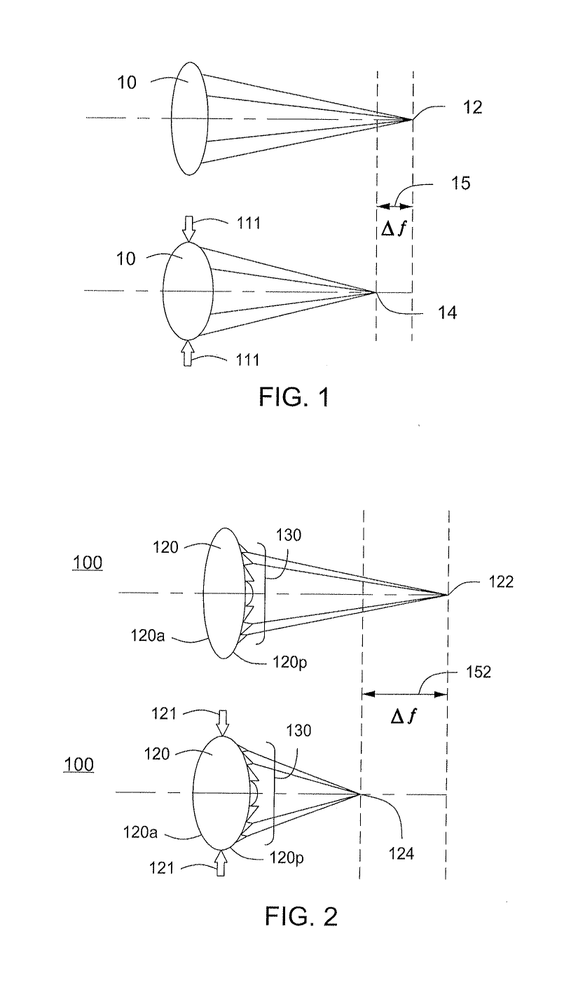

FIG. 2 depicts an exemplary embodiment of an accommodative IOL 100. The accommodative IOL 100 includes not only a deformable/accommodative lens 120, but also a kinoform-like diffractive grating pattern 130 on the posterior 120p of the lens 120. Note that when IOL 100 is compressed 121, the point of focus changes from 122 to 124, for a Δ_{f} 152, which is greater than Δ_{f} 150 (Δ_{f} 152>Δ_{f} 150). That is, the same amount of compression (ocular force) used on the IOL in FIG. 2 with the kinoform-like diffractive grating as on the IOL in FIG. 1 without kinoform-like diffractive grating results in a larger variation in power.

Typically the surface profile of the kinoform-like diffractive grating patterns includes a number of concentric radial rings. The area between rings is known as a zone, and the amount of power added to the lens by the diffractive element is determined in part by the diffractive grating pattern (e.g., the height and geometry of the diffractive grating pattern feature) and the width of the zone (e.g., length of the diffractive grating pattern feature).

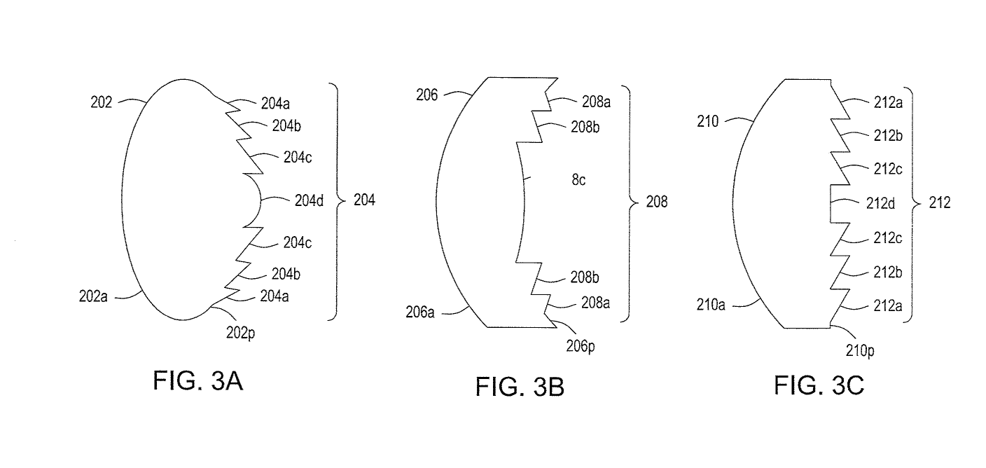

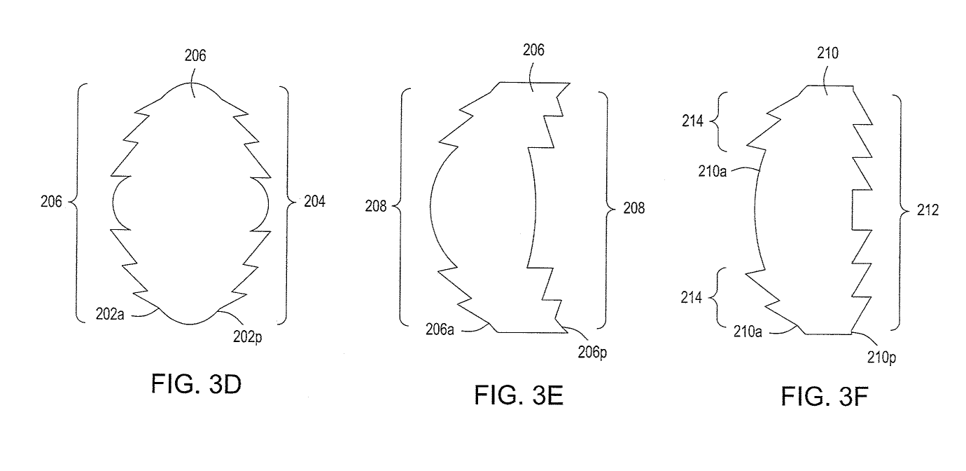

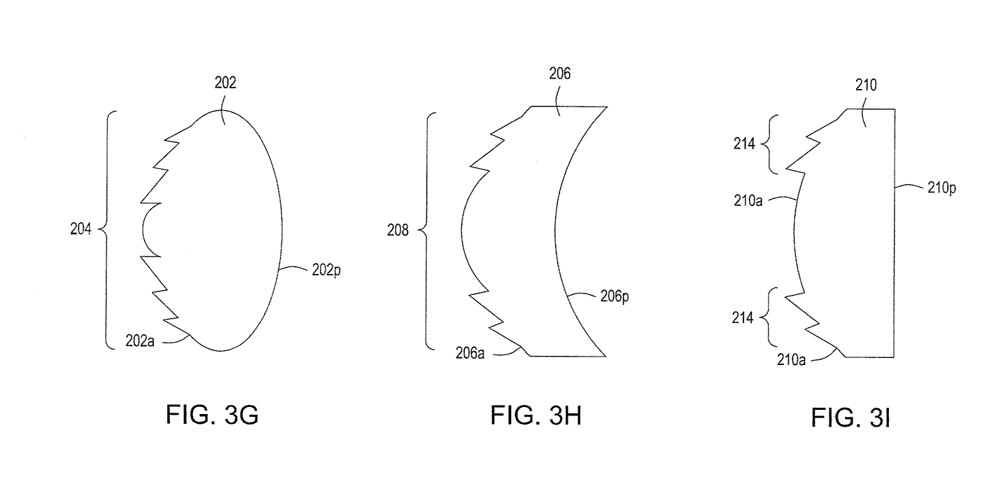

FIGS. 3A through 3I depict cross-sectional views of nine different exemplary embodiments of the accommodative IOL. FIG. 3A depicts an exemplary embodiment of an adaptive IOL including bi-convex lens 202, having an anterior surface 202a and a posterior surface 202p. A kinoform-like diffractive grating pattern 204 is disposed upon posterior surface 202p of lens 202. The kinoform-like diffractive grating pattern 204 comprises element 204a, element 204b, element 204c and a central element 204d. Kinoform-like diffractive grating pattern 204 is but one embodiment of the diffractive grating patterns usable in the accommodative IOL.

FIG. 3B depicts another exemplary embodiment of an accommodative IOL including a meniscus lens 206, having an anterior surface 206a and a posterior surface 206p. A kinoform-like diffractive grating pattern 208 is disposed upon posterior surface 206p of lens 206. The kinoform-like diffractive grating pattern 208 includes element 208a, element 208b, and a central element 208c. FIG. 3C depicts another exemplary embodiment of an accommodative IOL that is a plano-convex lens 210, having an anterior surface 210a and a posterior surface 210p. A kinoform-like diffractive grating pattern 212 is disposed upon posterior surface 210p of lens 210. The kinoform-like diffractive grating pattern 212 comprises element 212a, element 212b, element 212c; and central element 212d.

FIG. 3D depicts another exemplary embodiment of an accommodative IOL that includes a bi-convex lens 202, having an anterior surface 202a and a posterior surface 202p. The kinoform-like diffractive grating pattern 204 as in embodiment FIG. 3A is disposed upon posterior surface 202p of lens 202, but is also disposed on anterior surface 202a of lens 202. The kinoform-like diffractive grating pattern 204 on anterior surface 202a of lens 202 is a mirror image (through axis of symmetry 201) of the kinoform-like diffractive grating pattern 204 on posterior surface 202p of lens 202. However, in other embodiments, other symmetries may be possible.

FIG. 3E depicts another exemplary embodiment of an accommodative IOL that includes meniscus lens 206, having an anterior surface 206a and a posterior surface 206p. The kinoform-like diffractive grating pattern 208 as in embodiment FIG. 3B is disposed upon posterior surface 206p of lens 206, but is also disposed on anterior surface 206a of lens 206. The kinoform-like diffractive grating pattern 208 on anterior surface 206a of lens 206 is a not a mirror image of the kinoform-like diffractive grating pattern 208 on posterior surface 206p of lens 206 since lens 206 is vertically asymmetric.

FIG. 3F depicts another exemplary embodiment of an accommodative IOL that includes plano-convex lens 210, having an anterior surface 210a and a posterior surface 210p. The kinoform-like diffractive grating pattern 212, as in the embodiment of FIG. 3C, is disposed upon posterior surface 210p of lens 210. However, a different kinoform-like diffractive grating pattern 214 is disposed on anterior surface 210a of lens 210. FIG. 3F depicts another exemplary embodiment of an accommodative IOL that demonstrates that the kinoform-like diffractive grating pattern when present on both the anterior and posterior surfaces of an IOL may be different. In many embodiments the kinoform-like diffractive grating patters are different.

FIG. 3G depicts another exemplary embodiment of an accommodative IOL including a bi-convex lens 202, having an anterior surface 202a and a posterior surface 202p. A kinoform-like diffractive grating pattern 204 is disposed only upon the anterior surface 202a of lens 202. FIG. 3H depicts another embodiment of an accommodative IOL including a meniscus lens 206, having an anterior surface 206a and a posterior surface 206p. A kinoform-like diffractive grating pattern 208 is disposed only upon anterior surface 206a of lens 206. FIG. 3I depicts another embodiment of an accommodative IOL including a plano-convex lens 210, having an anterior surface 210a and a posterior surface 210p. A kinoform-like diffractive grating pattern 214 is disposed only upon anterior surface 210a of lens 210.

The kinoform-like diffraction grating may be applied to the lens in a number of different methods. For example, the diffractive grating may be integral with the anterior and/or posterior surfaces of the IOL as shown in embodiments FIG. 3A through FIG. 3I. Methods of manufacture to achieve an integral configuration include incorporating the diffractive grating into the pattern of the mold that is used to form the IOL; or in another method, the diffractive grating may be machined or etched into the anterior and/or posterior surfaces of the IOL after the IOL has been formed. In these embodiments, the material used to form both the main lens portion and the diffractive grating typically will be the same. Materials of used for the accommodative IOLs of the invention include but are not limited to silicones, acrylics (including, e.g., AcrySof®), and hydrogels.

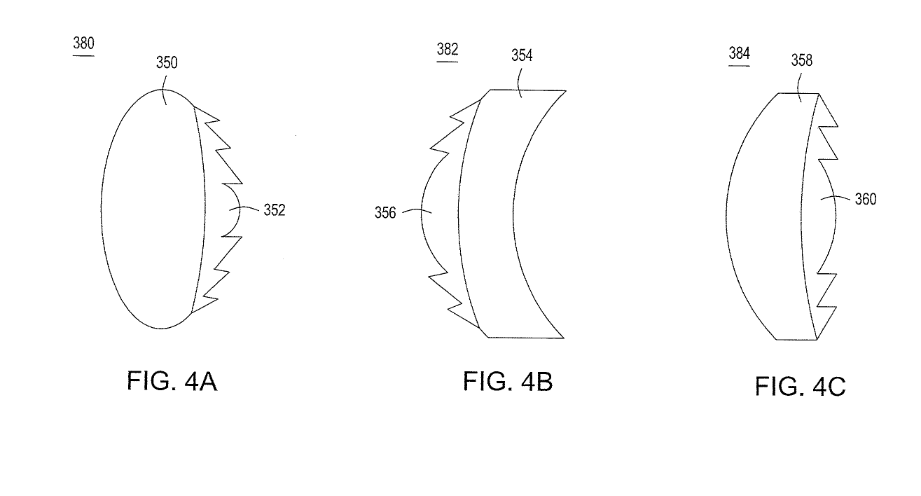

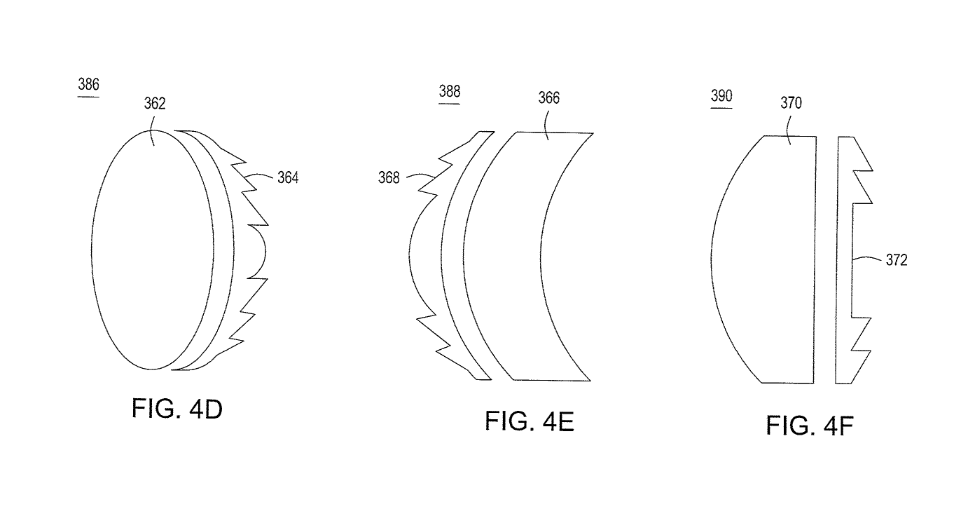

Alternatively, the diffractive grating may be fabricated separately from the IOL surface and then fastened or coupled to the anterior and/or posterior surfaces of the IOL after fabrication. In such embodiments, the diffractive grating may be fabricated of a different material than the main portion of the lens. FIGS. 4A through 4F depicts cross-sectional views of six different exemplary configurations for the accommodative IOL of the present invention, where the kinoform-like diffractive grating is not fabricated integral with the main portion of the lens.

FIG. 4A depicts an exemplary embodiment of an accommodative IOL 380 including a main bi-convex lens portion 350 and a diffraction grating portion 352 that has been coupled to the posterior surface of main lens portion 350. FIG. 4B depicts an exemplary embodiment of an accommodative IOL 382 including a main meniscus lens portion 354 and a diffraction grating portion 356 that has been coupled to the anterior surface of main lens portion 354. FIG. 4C depicts an exemplary embodiment of an accommodative IOL 384 comprising a plano-convex main lens portion 358 and a diffraction grating portion 360 that has been coupled to the posterior surface of main lens portion 358.

Embodiments FIG. 4D through 4F depict exemplary embodiments of accommodative IOLs in which the diffraction grating portion of element of the IOL is separate from the main lens portion of the accommodative IOL. FIG. 4D depicts an exemplary embodiment of an accommodative IOL 386 including a main bi-convex lens portion 362 and a diffraction grating portion 364 that is positioned behind but has not been coupled to the posterior surface of main lens portion 362. FIG. 4E depicts an exemplary embodiment of an accommodative IOL 388 including a main meniscus lens portion 366 and a diffraction grating portion 368 that is positioned in front of but has not been coupled to the anterior surface of main lens portion 366. FIG. 4F depicts an exemplary embodiment of an accommodative IOL 390 including a plano-convex main lens portion 370 and a diffraction grating portion 372 that is positioned in behind but has not been coupled to the posterior surface of main lens portion 370. The type of configuration shown in embodiments FIG. 4D through FIG. 4F may be applied where the diffractive grating portion may sit behind the capsular bag.

In addition to using the IOLs including kinoform-like diffractive grating patterns as described herein as a single IOL, the IOLs of the invention may be used in combination with other IOLs (e.g., one or more additional IOLs) to achieve the desired range of accommodation and power. For example, an IOL comprising a kinoform-like diffractive grating pattern made from silicone may be paired with (i.e., positioned anterior to or posterior to) a second lens made from acrylic.

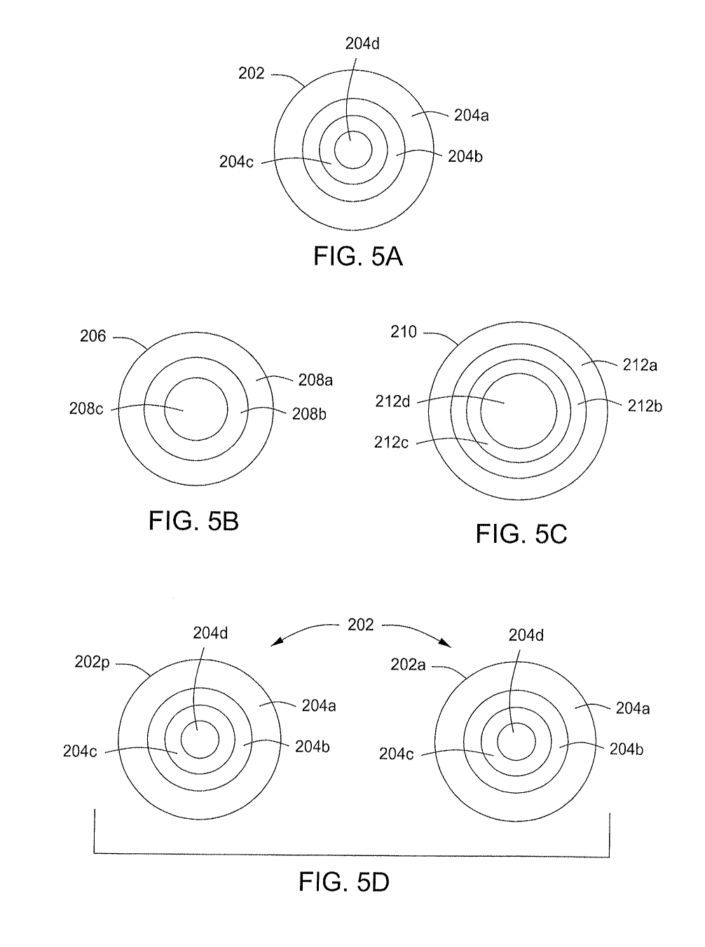

FIGS. 5A through 5D depict plan views of four of the exemplary configurations for the accommodative IOL of FIGS. 3A-3I. FIG. 54A is a plan view for, e.g., the posterior side of embodiment of the accommodative IOL depicted in FIG. 3A. FIG. 5B is a plan view for, e.g., the posterior side of the embodiment depicted in FIG. 3B. FIG. 5C is a plan view for, e.g., the posterior side of the embodiment depicted in FIG. 3C. FIG. 5D is a plan view for, e.g., the posterior and anterior side of the embodiment depicted in FIG. 5D.

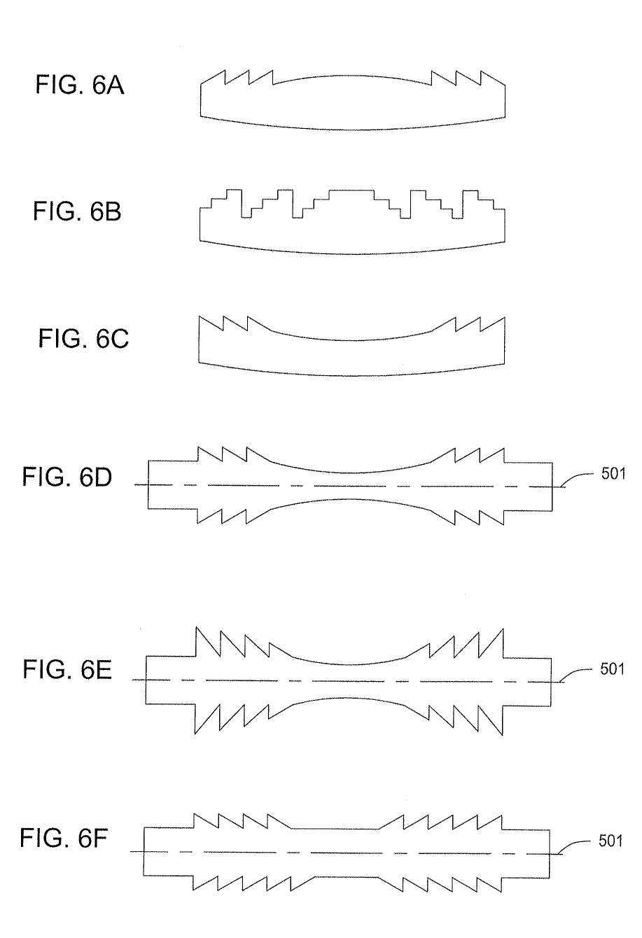

FIGS. 6A through 6F depict six exemplary kinoform-like diffractive grating patterns usable in the accommodative IOLs described herein. Kinoform-like diffractive grating patterns FIG. 6A through FIG. 6C may be us in accommodative IOLs including a kinoform-like diffractive grating pattern on only one of the anterior or posterior surface. Kinoform-like diffractive grating patterns FIG. 6D through FIG. 6F may be used in accommodative IOLs including a kinoform-like diffractive grating pattern on both the anterior or posterior surface. Kinoform-like diffractive grating patterns FIG. 6D and FIG. 6E demonstrate axes of symmetry through axes 501. In contrast, the kinoform-like diffractive grating pattern FIG. 6F is not symmetrical through axis 501.

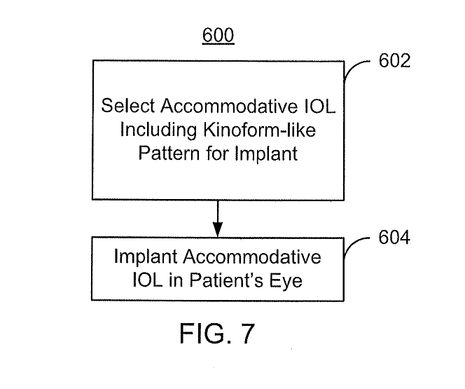

FIG. 7 is an exemplary embodiment of a method 600 for treating an ophthalmic condition in a patient. For simplicity, some steps may be omitted, interleaved, and/or combined. The method 600 is also described in the context of using the accommodative IOLs described herein.

An accommodative IOL, such as one or more of those described in FIGS. 1A, 2A-2I, 3A-3F, 4A-4D and 5A-5F, is selected for implantation in an eye of the patient is selected, via step 602. In some embodiments, another ophthalmic device such as another IOL may be used in conjunction with the accommodative IOL.

The selected accommodative IOL is implanted in the patient's eye, via step 604. Step 604 may include replacing the patient's own lens with the accommodative IOL or augmenting the patient's lens with the accommodative IOL. Treatment of the patient may then be completed. In some embodiments implantation in the patient's other eye of another analogous ophthalmic device may be carried out.

The preceding merely illustrates the principles of the invention. It will be appreciated that those skilled in the art will be able to devise various kinoform-like diffractive grating configurations or arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims. In the claims that follow, unless the term “means” is used, none of the features or elements recited therein should be construed as means-plus-function limitations pursuant to 35 U.S.C. §112, 6.

We claim:

1. A deformable intraocular lens configured to deform in response to an ocular force to provide accommodation, the deformable intraocular lens comprising:
an anterior surface;
a posterior surface disposed opposite the anterior surface; and
a kinoform-like diffractive grating pattern disposed on at least one of the anterior surface or the posterior surface, wherein the kinoform-like diffractive grating pattern varies under the ocular force resulting in enhanced power variation of the intraocular lens.

2. The deformable intraocular lens of claim 1 wherein the kinoform-like diffractive grating pattern is disposed on the anterior surface of the deformable intraocular lens.

3. The deformable intraocular lens of claim 1 wherein the kinoform-like diffractive grating pattern is disposed on the posterior surface of the deformable intraocular lens.

4. The deformable intraocular lens of claim 1 wherein the kinoform-like diffractive grating pattern is disposed on both the anterior and the posterior surface of the deformable intraocular lens.

5. The deformable intraocular lens of claim 1 wherein the lens is bi-convex in shape.

6. The deformable intraocular lens of claim 1 wherein the lens is meniscal in shape.

7. The deformable intraocular lens of claim 1 wherein the lens is plano-convex in shape.

8. The deformable intraocular lens of claim 1 wherein the kinoform-like diffractive grating pattern is integral to the deformable intraocular lens.

9. The deformable intraocular lens of claim 8 wherein the deformable intraocular lens is made from a silicone, a hydrogel or an acrylic.

10. The deformable intraocular lens of claim 9 wherein the deformable intraocular lens is made from AcrySof®.

11. The deformable intraocular lens of claim 1 wherein the kinoform-like diffractive grating pattern is not integral to the deformable intraocular lens.

12. The deformable intraocular lens of claim 11 wherein the deformable intraocular lens includes a main lens portion and a kinoform-like diffractive grating portion, and the main lens portion and the kinoform-like grating portion are made from different materials.

13. The deformable intraocular lens of claim 1 used in combination with at least one additional intraocular lens.

14. A deformable intraocular lens configured to deform in response to an ocular force to provide accommodation comprising:
an anterior surface;
a posterior surface disposed opposite the anterior surface; and
a kinoform-like diffractive grating pattern having at least two diffractive powers disposed on at least one of the anterior surface and the posterior surface, wherein the kinoform-like diffractive grating pattern stretches or shrinks under the ocular force resulting in power variation of the deformable intraocular lens.

15. The deformable intraocular lens of claim 14 wherein the kinoform-like diffractive grating pattern is disposed on both the anterior and the posterior surface of the deformable intraocular lens.

16. The deformable intraocular lens of claim 15 wherein the kinoform-like diffractive grating pattern disposed on the anterior surface of the lens is a mirror image through a vertical axis of symmetry to the kinoform-like diffractive grating pattern disposed on the posterior surface of the deformable intraocular lens.

17. The deformable intraocular lens of claim 15 wherein the kinoform-like diffractive grating pattern disposed on the anterior surface of the lens is not a mirror image through a vertical axis of symmetry to the kinoform-like diffractive grating pattern disposed on the posterior surface of the deformable intraocular lens.
